(19) United States
(12) Patent Application Publication
(10) Pub. No.: US 2019/0134331 A1
(43) Pub. Date: May 9, 2019

(54) RESPIRATOR HAVING IMPROVED SYNCHRONICITY DURING THE TRANSITION FROM EXPIRATORY TO INSPIRATORY OPERATION

(71) Applicant: Hamilton Medical AG, Bonaduz (CH)

(72) Inventors: Johannes Björn Thomas Meyer, Chur (CH); Dominik Novotni, Chur (CH)

(73) Assignee: Hamilton Medical AG, Bonaduz (CH)

(21) Appl. No.: 16/093,818

(22) PCT Filed: Apr. 10, 2017
(86) PCT No.: PCT/EP2017/058577

§ 371 Date: Oct. 15, 2018

(30) Foreign Application Priority Data
Apr. 15, 2016 (DE) 10 2016 206 442.8

(51) Int. Cl. A61M 16/00; A61M 16/10; A61M 16/20

(52) U.S. Cl. CPC A61M 16/026; A61M 16/104; A61M 16/208; A61M 16/0003; A61M 2230/46; A61M 2205/52; A61M 2205/505; A61M 2230/42; A61M 2016/0042

(57) ABSTRACT

A respirator for the at least supportive respiration of living beings includes a connection formation for connection to a breathing gas supply, a breathing gas conduit arrangement, pressure change arrangement for changing the pressure in the breathing gas conduit arrangement a flow sensor, which provides a flow signal representing a breathing gas volume flow in the breathing gas conduit arrangement, and a control unit controlling the operation of the respirator. The control unit is configured to trigger a transition from an expiratory to an inspiratory operation of the respirator whenever an increase in the steepness of a flow signal curve exceeds a steepness change threshold value.

The present invention relates to a ventilator for at least supportive ventilation of living beings breathing in a healthy state, the ventilator encompassing:
a connector configuration for connection to a respiratory gas reservoir;
a respiratory gas conduit arrangement conveying fresh inspiratory respiratory gas from the connector configuration to a patient interface upon inspiratory operation of the ventilator, and conveying metabolized expiratory respiratory gas away from the patient interface upon expiratory operation;
a pressure modifying arrangement for modifying the pressure of respiratory gas in the respiratory gas conduit arrangement;
a flow sensor that is embodied and arranged to furnish a flow signal representing a respiratory gas flow at least of expiratory respiratory gas in the respiratory gas conduit arrangement;
a control device that controls the operation of the ventilator and is embodied to trigger a transition from expiratory operation to inspiratory operation of the ventilator when an increase in the slope of a flow signal profile, which reproduces flow signals furnished successively in time, exceeds a slope change threshold value.

A ventilator of this species is known from EP 0 521 314 A1. The teaching of this document is to switch control of a ventilator between an expiratory phase and an inspiratory phase when the respiratory flow curve ascertained during the ventilation phase exhibits a significant increase in its slope. A determination as to whether or not such a significant increase exists is made based on a comparison between a slope increase that occurs and a predetermined slope change threshold value. This teaching represents an improvement with respect to other trigger criteria, likewise known from the existing art, for a transition from expiratory to inspiratory operation of the ventilator.

One very simple trigger criterion is represented by the baseline-level crossing, for example a zero crossing or a crossing through a baseline level different from zero, of the respiratory flow curve, i.e. of the time-related flow signal profile. This trigger criterion is known, for example, from U.S. Pat. No. 3,834,382. A disadvantage of this criterion is that it functions satisfactorily essentially only in patients having a healthy respiratory system, whereas during artificial ventilation of patients with chronic obstruction (COPD patients), asynchronicities that are unpleasant for the patient can occur if the patient exhibits spontaneous respiration during artificial ventilation.

The trigger criterion of a baseline-level crossing of the flow signal is furthermore sensitive to leakage-related effects that can occur in the context of artificial ventilation, for example because a ventilator mask, constituting one possible patient interface, is not completely sealing against the facial region around the nose and mouth of the patient to be ventilated. There can also be other causes of leakage flows, and thus leakage-related effects, during artificial ventilation.

In order to avoid leakage-related errors in ETS-based triggering, the teaching of DE 44 32 219 C1 is to raise a respiratory gas flow trigger threshold for a transition from expiratory operation to inspiratory operation by an additional amount that is calculated from the ratio between the average respiratory pressure during inspiration and the sum of the averaged respiratory pressures during inspiration and expiration, multiplied by the difference between the averaged values of the respiratory flows during inspiration and during expiration.

The ETS-based technical teaching known from DE 44 32 219 C1 can, however, also result in undesired asynchronicities during artificial ventilation for patients having pathological respiratory organs, for example COPD patients.

It is also known from U.S. Pat. No. 6,439,229 B1 to modify ETS-based decision thresholds for so-called “cycling,” i.e. for a transition from inspiratory to expiratory operation of the ventilator, as a function of the expiratory time constant of the respective patient being ventilated.

“ETS” refers to “expiratory trigger sensitivity,” which is a percentage of the maximum respiratory gas flow occurring during an inspiration event, which serves as a cycling threshold value. When the respiratory gas flow has dropped to the predetermined cycling threshold value during the inspiration phase, the ventilator is switched over from inspiratory operation to expiratory operation. For healthy patients, the ETS-based cycling threshold value is often approximately 25% of the maximum respiratory gas flow occurring during the inspiration phase.

The teaching of U.S. Pat. No. 6,439,229 B1 is to preselect a range of possible cycling threshold values as a function of the ascertained expiratory time constant of the respective patient being ventilated and to select within the preselected percentage range, as a function of the magnitude of a pressure overshoot (referred to as a “supra-plateau pressure”) in the respiratory gas pressure profile at the end of the pressure-assisted inspiration phase, a specific percentage value of the maximally occurring inspired respiratory gas flow as a cycling threshold value.

With the technical teaching known from U.S. Pat. No. 6,439,229 B1 it is possible to improve the synchronicity of artificial ventilation using a ventilator on a spontaneously breathing patient, but because of the ETS-based control behavior of the known ventilator it is susceptible to leakage flows that often occur; this can then negate the synchronicity advantage resulting from adaptation of the cycling threshold value based on the expiratory time constant and the above-described pressure behavior.

A reduced susceptibility to leakage flows compared with ETS-based triggering thresholds and cycling thresholds is obtained with the technical teaching known from EP 0 521 314 A1 of the species, since what is important for triggering a transition from expiratory to inspiratory operation is no longer a specific value or specific value ratio of the respiratory gas flow, but instead the shape of the time-related respiratory gas flow signal profile. This shape is retained within certain limits even if leakage occurs. A ventilator operating according to the teaching of EP 0 521 314 A1 can ventilate even COPD patients with improved synchronicity compared with other ventilators that operate, for example, in ETS-based fashion. COPD is, however, only one pathological change that can occur in the respiratory system. Using the technical teaching known from EP 0 521 314 A1, patients having other respiratory system changes that also occur, and that can result in respiratory behavior that deviates from healthy respiratory behavior, may in fact be ventilated with synchronicity that is worse than with ventilators operating on an ETS basis.

In addition, when both COPD patients and patients having an unimpaired respiratory system are to be ventilated using one and the same ventilator operating in accordance with EP 0 521 314 A1, the predetermined slope change threshold value used in that context is always a compromise that is intended to be applicable to all patients and can thus result in asynchronicities in ventilation in individual cases, since the respective patient to be ventilated is consistent only to a limited extent with the situation on which the predetermined slope change threshold value is based.

The object of the present invention is therefore to improve the ventilator recited previously in such a way that with it, patients can be ventilated with improved synchronicity regardless of the pathological state of their respiratory system.

The present invention achieves this object by way of a ventilator of the species in which the control device is further embodied to ascertain the slope change threshold value as a function of an expiratory time constant of the respective patient to be ventilated, and to trigger the transition from expiratory operation to inspiratory operation of the ventilator when the increase in the slope of the flow signal profile exceeds the slope change threshold value ascertained in accordance with the expiratory time constant.

By ascertaining, and if applicable modifying and thus adapting, the slope change threshold value as a function of the expiratory time constant of the respective patient to be ventilated, it is possible to adapt the transition from expiratory operation to inspiratory operation of the ventilator almost optimally to the pathological state of the respiratory system of the respective patient, since the expiratory time constant of a person represents a parameter that is highly relevant to artificial ventilation of a patient.

Based on the change in the slope change threshold value as a function of the expiratory time constant of the respective patient to be ventilated, operation of the ventilator at least in the context of the transition from expiratory to inspiratory operation can thus be adapted to any pathological states of patients' respiratory systems.

To clarify: the term “triggering” refers in the present Application only to the initiation of a transition from expiratory operation to inspiratory operation of the ventilator. Conversely, the term “cycling” is used for the initiation of a transition in the opposite direction, from inspiratory to expiratory operation.

With the control device embodied according to the present invention it is possible to ventilate with outstanding synchronicity not only COPD patients, who exhibit a disproportionately high expiratory time constant compared with patients having healthy respiratory systems, but also ARDS patients, whose expiratory time constant is disproportionately short compared with patients having healthy respiratory systems.

One asynchronicity that often occurs in the context of the transition under discussion here (from expiratory to inspiratory operation) is so-called “auto-triggering,” in which, because of correspondingly preset decision criteria, the ventilator initiates an inspiration event even though the patient is not actually inhaling or making efforts toward inspiration. The situation that then occurs is that the ventilator delivers respiratory gas to the patient, who in fact still wishes to exhale or is at least about to exhale, against his or her respiratory efforts. This can produce in patients, despite sedation, considerable reactions that are also disadvantageous for further artificial ventilation.

Because of the obstructive respiratory behavior, COPD, which is also commonly referred to as “asthma” or “smoker's lung,” results in comparatively steady respiratory gas flow signal profiles in which a change in the magnitude of their slope can be identified relatively easily and reliably, so that even the previously known ventilator of the species provides comparatively good results for COPD patients.

ARDS patients have a “restructive” respiratory system, commonly referred to as a “hard lung.” Respiration occurs in this case in pulses, which can result, during an expiration phase, in one or more discontinuities in the form of kinks in the respiratory gas flow signal profile. These kinks represent changes in the magnitude of the slope of the flow signal which can cause auto-triggering when a slope change threshold value is disadvantageously selected.

The present invention also helps ARDS patients to be ventilated with improved synchronicity using the ventilator improved according to the present invention.

Given the correlation mentioned, it is advantageous to ventilate ARDS patients, whose expiratory time constant is shorter than that of patients having a healthy respiratory system, using a slope change threshold value of a higher magnitude, so that discontinuities in the flow signal profile during an expiration phase, which are typical of ARDS, are not sensed by way of the slope change threshold value and so that, correctly, what is utilized as a trigger signal is only a change of greater magnitude in the slope of the flow signal profile at the end of the expiration phase, when a transition from expiratory to inspiratory operation should in fact take place in accordance with the patient's behavior.

COPD patients, whose expiratory time constant is longer than that of patients having a healthy respiratory system, can likewise be ventilated using a slope change threshold value that is lower in magnitude than that of ARDS patients, and lower than that of patients having a healthy respiratory system.

The control device is therefore preferably embodied to decrease the magnitude of the slope change threshold value with an increasing expiratory time constant, and vice versa.

To avoid undesired incorrect triggering, the control device is preferably embodied to examine for the presence of an exceedance of the slope change threshold value only those changes in the slope of the flow signal profile which cause the slope of the flow signal profile to increase, i.e. cause the flow signal profile to rise progressively. The slope change threshold value is then preferably a slope increase threshold value, to be utilized in rising portions of the slope signal profile but not in falling ones.

To clarify further at this juncture: the trigger criterion discussed in the present Application is not the only trigger criterion according to which the control device modifies, or can modify, operation of the ventilating apparatus from expiratory to inspiratory. The control device preferably has a plurality of trigger criteria, for example more than 25, in the presence of each of which the control device causes operation of the ventilating apparatus to switch over from expiratory to inspiratory.

In principle, according to a simple exemplifying embodiment of the present invention, provision can be made that the control device interacts with an input apparatus, for example a keyboard and the like, by way of which it is possible to enter an expiratory time constant relevant to the respective patient to be ventilated.

The aforementioned flow sensor already makes it possible in principle to ascertain the expiratory time constant of a patient to be ventilated, and for that reason the control device is preferably embodied to ascertain the expiratory time constant in one respective ventilation use instance. The flow sensor preferably senses the respiratory gas flow as a volumetric flow. Additional or alternative sensing as a mass flow is likewise possible.

It is conceivable for the time constant to be ascertained once at the beginning of the ventilation use instance for the entire remaining duration of the ventilation use instance, and to carry out the ventilation use instance using the ascertained value.

Alternatively, it is conceivable to ascertain the expiratory time constant repeatedly at predetermined time intervals during a ventilation use instance, and to continue the ventilation use instance using the respective most recently ascertained time constant value. Particularly accurate, and therefore advantageously particularly synchronous, ventilation of a patient can be achieved by the fact that the control device is embodied to ascertain the expiratory time constant breath by breath.

In principle, there are numerous known possibilities for automatically ascertaining a patient's expiratory time constant. Particularly advantageous methods for this are disclosed in Josef X. Brunner et al., “Simple method to measure total expiratory time constant based on the passive expiratory flow-volume curve,” in Critical Care Medicine, 1995, Vol. 23, No. 6, pp. 1117 ff., the content of which is referred to here.

For example, the control device can be embodied to ascertain the expiratory time constant, preferably iteratively numerically, in accordance with the ratio between the volume exhaled during an expiration event and the maximum expiratory volumetric flow that occurred in that context, and/or in accordance with the resistance and compliance that occur in the context of one breath. Ascertaining the resistance and compliance separately, and calculating the expiratory time constant therefrom by multiplication, is possible but is less preferred than calculating the time constant from the ratio of exhaled volume to the maximum expiratory volumetric flow that occurred in that context. Iterative numerical calculation methods exist here which converge after only a few iteration steps to a relevant value, which differs to only a negligible degree from the actual time constant for the respective ventilation instance.

The term “flow signal profile” is not to be understood to mean that the entire time profile of the flow signal furnished by the flow sensor must be recorded and furnished. In order to achieve the advantages of the present invention it is sufficient if the flow signal profile exists over a sufficiently long time span that the change in a slope of the flow signal profile can be ascertained therefrom. The term “flow signal profile” is also not to be misunderstood to mean that a continuous profile is obligatorily required in order to ascertain a change in its slope. A sequence of chronologically successive discrete flow signal values also represents a flow signal profile for purposes of the present Application. For example, three flow signal values ascertained in chronological succession are sufficient for ascertaining and evaluating a change in the slope of that short flow signal profile. Between a first and a second flow signal value, for example, a slope of the flow signal profile between those two interpolation points can be ascertained by means of a difference quotient, and likewise between a second and a third flow signal value, once again using a difference quotient. The slopes thereby ascertained, firstly between a first and a second flow signal value and again between a second and a third flow signal value, can be compared with one another, for example by subtraction, and the difference value, which represents an indication of the change in the steepness or slope of the flow signal profile, can be compared with the slope change threshold value of the flow signal profile. Triggering can be caused, or not, depending on the outcome of such a comparison with the slope change threshold value.

When “chronological successively” ascertained or furnished flow signal values are referred to in the present Application, this preferably, but not necessarily, means that they are ascertained or furnished immediately successively.

Further possibilities for ascertaining the change in the slope of a flow signal profile are of course known. For example, the flow signal profile can be approximated at least in portions by a function; that approximation function can be derived twice with respect to time. The second derivative of a function is known to be an indication of the change in the slope of a function or of a value profile.

For useful modification of the slope change threshold value on the basis of the expiratory time constant of the patient to be ventilated, according to an advantageous refinement of the present invention provision can be made that the ventilator comprises a data memory, at least readable by the control device, in which a correlation between the magnitude of the slope change threshold value and the expiratory time constant, or a correlation between a change value of the magnitude of the slope change threshold value and the expiratory time constant, is stored. The correlation can also, by way of the magnitude of the slope change threshold value, relate to its sign.

The correlation can be stored in the form of a tabular or graphic characteristics diagram. Preferably, a functional correlation between the magnitude of the slope change threshold value and the expiratory time constant, or a functional correlation between a change value of the magnitude of the slope change threshold value and the expiratory time constant, is stored in the data memory, depending on whether the slope change threshold value is to be ascertained absolutely or whether only a change magnitude that is to be applied to the previously used slope change threshold value is to be ascertained, i.e. whether the change in the slope change threshold value is to occur absolutely or incrementally. Both are possible in principle. The control device is then preferably embodied to ascertain, proceeding from the expiratory time constant on the basis of the functional correlation, a slope change threshold value that is to be used. There is thus no need to calculate an interpolation or extrapolation of slope change threshold values, but instead the slope change threshold value respectively relevant to a time constant value can be ascertained directly, optionally applying the intermediate step of ascertaining a change increment and applying the change increment to the most recently valid slope change threshold value.

The ventilator preferably comprises a data memory to which the control device can write and in which flow signal values furnished by the flow sensor can be stored, in order to ascertain a time-related flow signal profile from the values. This can be the data memory referred to above.

The flow signal received from the flow sensor is often noisy and therefore less than optimally suitable for immediate evaluation. Provision can therefore further be made that the control device is embodied to smooth the flow signal furnished by the flow sensor, for example by low-pass filtering or by calculation of a moving average. The calculation of a moving average can take into account the five to six most recent flow signal values, optionally weighted. Smoothing of the time-related flow signal profile is advantageous principally when the change in the slope of the flow signal profile is to be effected by differentiation of an approximation function that is approximated at least in portions to the flow signal profile, since an approximation function for a smooth signal profile can be ascertained with little error. The expiratory time constant is preferably ascertained on the basis of the smoothed flow signal profile.

In order to avoid undesired triggering due to changes in the slope of the flow signal profile in the initial phase of expiratory operation, according to an advantageous refinement of the present invention the control device is embodied not to trigger the transition from expiratory operation to inspiratory operation of the ventilator until a predetermined time span has elapsed after a predefined characteristic event, for example a beginning of expiratory operation or a baseline-level crossing of the flow signal. The baseline-level crossing of the flow signal can be a crossing through a zero level or through a baseline level differing from zero.

At the beginning of expiratory operation, usually an inspiration valve of the ventilator is closed and an expiration valve is opened, with the result that a change in the direction of the respiratory gas flow at the patient occurs. The sign of the flow signal changes in this context, i.e. its magnitude at first decreases sharply and then rises sharply with an opposite sign. Proceeding from the maximum respiratory gas flow that then occurs, the magnitude of the flow signal decreases again as expiration proceeds. A change in the slope of the flow signal profile therefore occurs as a rule at the beginning of an expiration phase, but this is specifically not intended to trigger a transition from expiratory to inspiratory operation. This is prevented by the feature discussed above.

In this context as well, it is conceivable for the predetermined time span after occurrence of the characteristic event, within which span a transition is not to be triggered, to be ascertained on the basis of the expiratory time constant, since the expiration phase lasts longer in COPD patients than in patients having a healthy respiratory system, and the expiration phase is shorter in ARDS patients than in patients having a healthy respiratory system.

The predetermined time span can therefore be selected to be shorter for ARDS patients than for patients having a healthy respiratory system, and their time span can in turn be shorter than that of COPD patients. The predetermined time span can therefore be selected, for example, proportionally to the expiratory time constant.

The ventilator according to the present invention is preferably embodied for artificial ventilation in pressure support ventilation (PSV) mode.

The present invention will be explained below in further detail with reference to the appended drawings, in which:

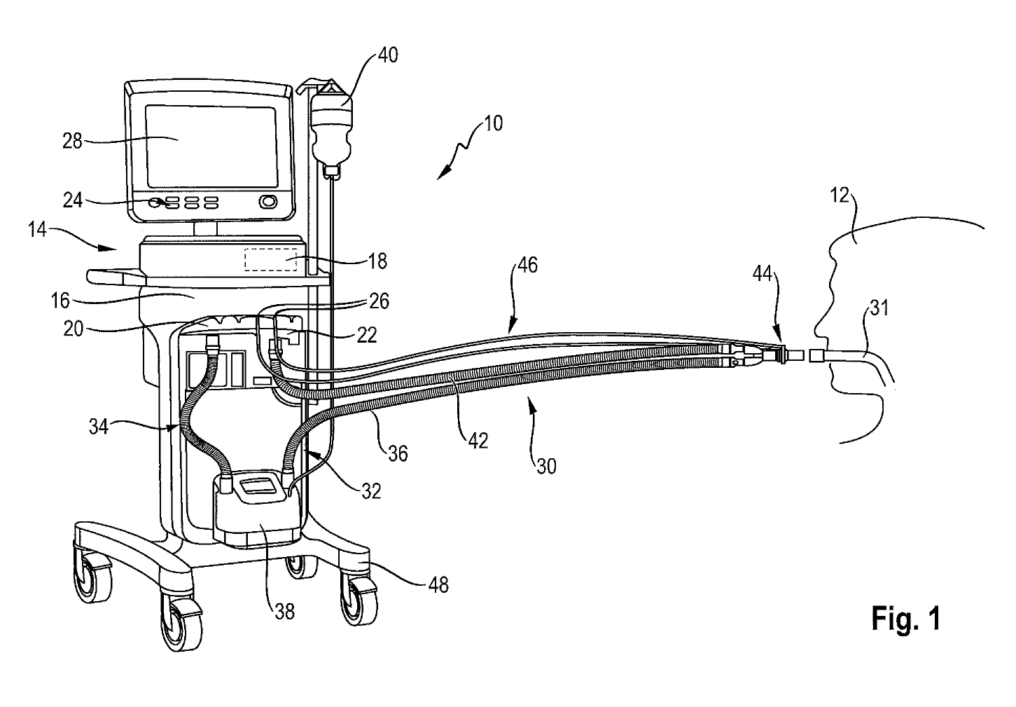

FIG. 1 schematically depicts a ventilation apparatus according to the present invention, configured for artificial ventilation of a patient;

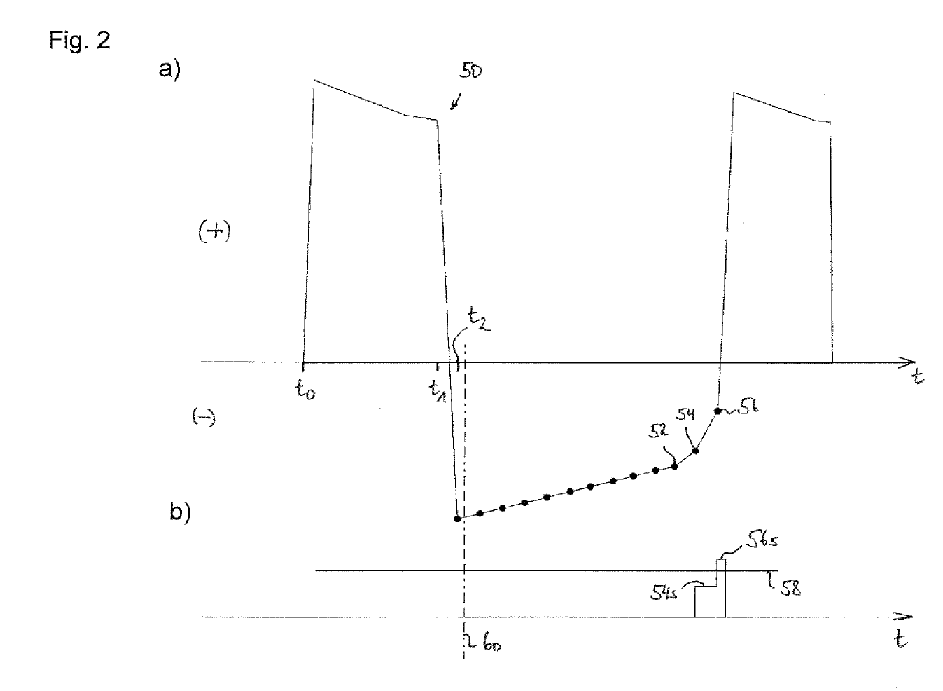

FIG. 2a shows an example of a time profile of a flow signal of a COPD patient;

FIG. 2b is an exemplifying graphic depiction of increases occurring, in the time-related flow signal profile of FIG. 2a, in the slope of the flow signal profile, with an applicable slope change threshold value for triggering an inspiration event;

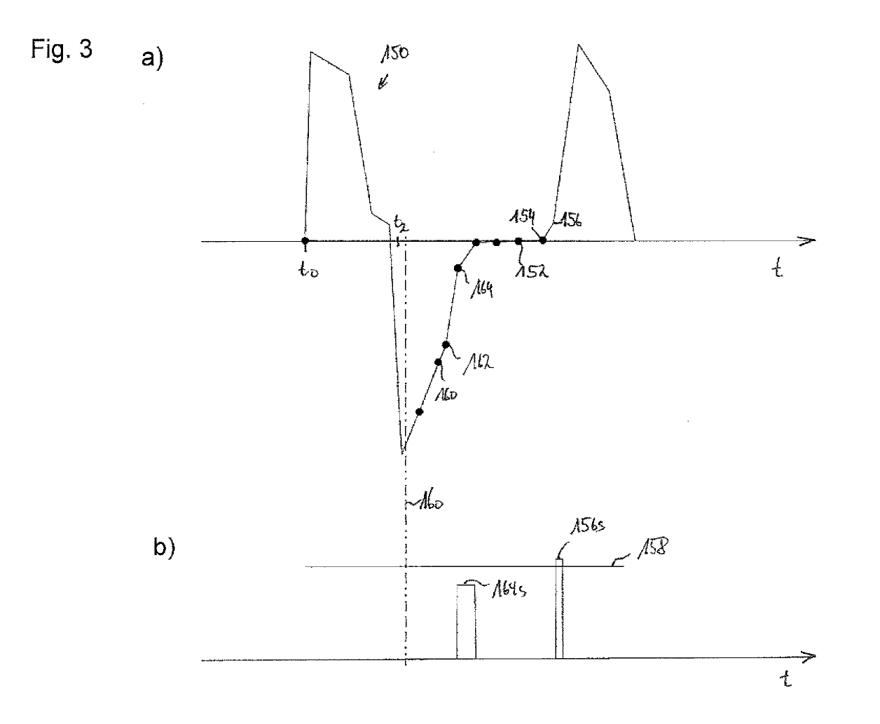

FIG. 3a shows an example of a flow signal of an ARDS patient; and

FIG. 3b is an exemplifying graphic depiction of increases occurring, in the time-related flow signal profile of FIG. 3a, in the slope of the flow signal profile, with an applicable slope change threshold value for triggering an inspiration event.

In FIG. 1, an embodiment according to the present invention of a ventilation apparatus is labeled in general with the number 10. In the example depicted, ventilation apparatus 10 serves for artificial ventilation of a human patient 12 in PSV mode.

Ventilation apparatus 10 has a housing 14 in which a pressure modification arrangement 16 and a control device 18 (not visible from outside because of the opaque housing material) can be received.

Pressure modification arrangement 16 is constructed in a manner known per se and can comprise a pump, a compressor, a fan, a pressure vessel, a reducing valve, and the like. Ventilation apparatus 10 furthermore comprises, in a manner known per se, an inspiration valve 20 and an expiration valve 22.

Control device 18 is usually implemented as a computer or microprocessor. It encompasses a memory device (not depicted in FIG. 1) so that data required for the operation of ventilation apparatus 10 can be stored and retrieved as necessary. In a context of network operation, the memory device can also be located outside housing 14 and can be connected to control device 18 by a data transfer connection. The data transfer connection can be constituted by a cable link or radio link. In order to prevent interference on the data transfer connection from being able to affect the operation of ventilation apparatus 10, however, the memory device is preferably integrated into control device 18 or at least received in the same housing 14 with it.

For inputting data into ventilation apparatus 10 or more precisely into control device 18, ventilation apparatus 10 can have a data input 24 that is represented, in the example depicted in FIG. 1, by a keyboard. As will be further explained below, the keyboard is not the only data input of control device 18. Control device 18 can in fact obtain data via various data inputs, for example via a network line, a radio link, or via sensor terminals 26 that will be discussed individually later on.

In order to output data to the person performing treatment, ventilation apparatus 10 can comprise an output device 28, in the example depicted a screen.

For artificial ventilation, patient 12 is connected to ventilation apparatus 10, more precisely to pressure modification arrangement 16 in housing 14, via a respiratory gas conduit arrangement 30. Patient 12 is intubated for that purpose. Tube 31 constitutes a patient interface of ventilator 10. Alternatively, the patient interface can encompass a mask covering the nose and mouth.

Ventilator 10 furthermore comprises a connector configuration (not depicted in FIG. 1) for connection to a respiratory gas reservoir. In a simple case, this connector configuration can be an aspiration tube through which ambient air can be aspirated—with interposition of a filter, if desired—from the immediate environment of ventilator 10 into respiratory gas conduit arrangement 30. The connector configuration can also be a flow conduit coupling by means of which the ventilator is connectable to a reservoir of gas (either air or oxygen). The gas reservoir can be, for example, a gas vessel or a collector reservoir that is installed as part of a hospital's building technology.

Respiratory gas conduit arrangement 30 comprises an inspiration hose 32 through which fresh respiratory gas can be directed from pressure modification arrangement 16 into the lungs of patient 12. Inspiration hose 32 can be interrupted, and can comprise a first inspiration hose 34 and a second inspiration hose 36 between which a conditioning device 38 can be provided for controlled humidification and, if applicable, also temperature control of the fresh respiratory gas delivered to patient 12. Conditioning device 38 can be connected to an external liquid reservoir 40 by way of which water for humidification or also a medication, for example to inhibit inflammation or to expand the airways, can be delivered to conditioning device 38. When the present ventilation apparatus 10 is used as an anesthesia ventilation apparatus, volatile anesthetics can thereby be conveyed in controlled fashion via ventilation apparatus 10 to patient 12. Conditioning device 38 ensures that the fresh respiratory gas is conveyed to patient 12 with a predetermined moisture content, if applicable with addition of a medication aerosol, and at a predetermined temperature.

In addition to inspiration valve 20 and expiration valve 22 already mentioned, respiratory gas conduit arrangement 30 furthermore comprises an expiration hose 42 through which metabolized respiratory gas is discharged from the lungs of patient 12 into the atmosphere.

Inspiration hose 32 is coupled to inspiration valve 20, and expiration hose 42 to expiration valve 22. Only one of the two valves is respectively opened at one time to allow passage of a flow of gas. Actuation control of valves 20 and 22 is also accomplished by control device 18.

During a respiration cycle, firstly expiration valve 22 is closed and inspiration valve 20 opened for the duration of the inspiration phase, so that fresh respiratory gas can be directed from housing 14 to patient 12. A flow of fresh respiratory gas is brought about by controlled elevation of the respiratory gas pressure by pressure modification arrangement 16. As a result of the pressure elevation, fresh respiratory gas flows into the lungs of patient 12 where it expands the region of the body close to the lungs, i.e. in particular the ribcage, against the individual elasticity of those parts of the body close to the lungs. The gas pressure in the interior of the lungs of patient 12 also rises as a result.

At the end of the inspiration phase, inspiration valve 20 is closed and expiration valve 22 is opened. The expiration phase begins. Because the gas pressure of the respiratory gas present in the lungs of patient 12 has been elevated up to the end of the inspiration phase, that gas flows into the atmosphere after expiration valve 22 is opened, and the gas pressure in the lungs of patient 12 decreases as the flow duration progresses. When the gas pressure in lungs 12 reaches, for example, a positive final-expiration pressure set on ventilation apparatus 10, i.e. a pressure slightly higher than atmospheric pressure, the expiration phase is terminated with the closing of expiration valve 22, and a further respiration cycle follows. This can be one of several trigger criteria whose presence allows the control device to trigger a switchover of the ventilator from expiratory to inspiratory operation.

During the inspiration phase, what is delivered to patient 12 is the so-called ventilation tidal volume, i.e. the volume of respiratory gas per breath. The ventilation tidal volume multiplied by the number of ventilation cycles per minute, i.e. multiplied by the ventilation frequency, yields the volume per minute of artificial ventilation currently being carried out.

Ventilation apparatus 10, in particular control device 18, is preferably embodied to repeatedly update or ascertain, during ventilation operation, the ventilation operation parameters that characterize ventilation operation of ventilation apparatus 10, in order to ensure that ventilation operation is coordinated as optimally as possible at every point in time with the respective patient 12 to be ventilated. Particularly advantageously, the one or several ventilation operating parameters are determined at the ventilation frequency, so that up-to-date ventilation operating parameters, which are thus optimally adapted to patient 12, can be furnished for each ventilation cycle.

Ventilation apparatus 10 is connected in data-transferring fashion at least to a flow sensor 44, preferably also to further sensors such as a pressure sensor to measure the respiratory gas pressure in respiratory gas conduit arrangement 30. Flow sensor 44 senses the respiratory gas flow present in respiratory gas conduit arrangement 30 and outputs a signal that represents the respiratory gas flow. Flow sensor 44 is preferably coupled by means of a sensor line arrangement 46 to data inputs 26 of control device 18. Sensor line arrangement 46 can, but does not need to, encompass electrical signal transfer leads. It can also comprise hose lines that transfer to data inputs 26 the gas pressure present in a flow direction on either side of flow sensor 44, where they are quantified by pressure sensors (not depicted in FIG. 1).

Merely for the sake of completeness, be it noted that ventilation apparatus 10 according to the present invention, constituting a mobile ventilation apparatus 10, can be received on a rolling frame 48.

Flow sensor 44 is preferably arranged in a portion of respiratory gas conduit arrangement 30 in which it can sense both the inspiratory flow and the expiratory flow. Alternatively, or also additionally for reasons of redundancy, at least one further flow sensor can be provided respectively in inspiration hose 32 and/or in expiration hose 42 and/or in housing 14.

FIG. 2a depicts, schematically and by way of example, a time-related flow signal profile 50 that is obtained when patient 12 of FIG. 1 is a COPD patient.

The inspiration event begins at time t₀, at which control device 18 initiates the above-described inspiration event and elevates the pressure in inspiration line 32 to a preset inspiration pressure.

As a result of the pressure elevation in inspiration hose 32, the flow of respiratory gas, indicated e.g. as a volumetric flow in volume per unit time, rises rapidly and then gradually decreases in magnitude as the lungs increasingly fill with fresh respiratory gas. By convention, the flow of respiratory gas to or into patient 12 is considered to have a positive sign.

As the lungs of patient 12 fill, his or her lungs, and with them the patient's respiratory musculature and ribcage surrounding them, become expanded. At a time t₁ a cycling takes place, i.e. a switchover of ventilator 10 from inspiratory operation to expiratory operation. Inspiration valve 20 is closed, and expiration valve 22 is opened. The result is that the flow of respiratory gas to the patient decreases abruptly and its flow direction reverses, driven by the body pressure in the region of the respiratory organs of patient 12 which was previously elevated by the introduction of fresh respiratory gas into the lungs. Exhalation on the part of patient 12 occurs passively, by the fact that the body parts expanded by the influx of fresh respiratory gas are allowed to relax.

The flow of respiratory gas becomes negative due to the reversal of direction; it is only at time t₂ after switchover of the valves (inspiration valve 20 and expiration valve 22) that the actual expiration event begins, in which the metabolized respiratory gas escapes from the lungs of patient 12. In COPD patients this occurs only gradually because of their obstructive respiratory system, so that flow signal profile 50 increases with a comparatively shallow and initially constant slope. The flow of respiratory gas decreases in magnitude, but in consideration of the sign of the flow it increases, since it is changing toward lower negative absolute values. This means that per unit time, the metabolized respiratory gas expired by the COPD patient decreases by an approximately constant volumetric flow difference amount.

It is only as emptying of the lungs of COPD patient 12 progresses that changes, in particular increases, in the slope of flow signal profile 50 occur. Reference may be made here to the last three interpolation points of the flow signal profile, i.e. points 52, 54, and 56.

As is evident from FIG. 2a, the magnitude of the slope or steepness of flow signal profile 50 changes between interpolation points 52 and 54 as compared with the points preceding point 52. The slope increases.

The steepness or slope of the flow signal profile changes again between interpolation points 54 and 56. The slope again increases.

In FIG. 2b, the increase in slope is plotted against a time axis t correlated with the time axis t of FIG. 2a. Corresponding to the depiction of the flow signal profile in FIG. 2a in the region of interpolation points 52 to 56, the change in the slope of flow signal profile 50 from interpolation point 52 to interpolation point 54 as compared with the preceding profile of the curve is plotted to be less than the change in the slope of profile 50 between interpolation points 54 and 56. The changes in slope are respectively associated with the chronologically later interpolation points of an interpolation point pair, i.e. points 54 and 56, utilized for calculation of the slope. The change value, in particular increase value, of the slope of flow signal profile 50 in the region of interpolation points 52 and 54, compared with a profile portion located earlier, is therefore labeled 54s, and the change value, in particular increase value, of the flow signal profile in the region of interpolation point pair 54 and 56, relative to the portion between interpolation point pair 50 and 52, is labeled 56s.

Also plotted in FIG. 2b is a slope change threshold value 58 that is utilized as a trigger criterion for initiating a transition from expiratory operation to inspiratory operation of ventilation device 10.

In the example of FIG. 2b, slope change threshold value 58 is selected with reference to COPD patients so that change value 54s, at which the patient is still in the expiration phase and has not made any independent effort toward inspiration, is below slope change threshold value 58.

Change value 56s, however, is based on a value typical of COPD patients upon exertion of an independent inspiration effort, so that this value exceeds slope change threshold value 58 and thus causes control device 18 to switch ventilator 10 from expiratory operation to inspiratory operation.

As is evident from FIG. 2b, a massive increase in the slope of flow signal profile 50 occurs in the initial phase of expiration, namely at time t₂, when after an initial sharp rise in the magnitude of the expiration flow, the flow of respiratory gas out of the patient gradually decreases again in magnitude.

To prevent this change in the slope of flow signal profile 50 from initiating a transition to inspiratory operation already at the beginning of expiratory operation, a time threshold 60 is stored in control device 18; only beyond that threshold is a change in the slope of the time-related flow signal profile 50 responded to for provision of a trigger signal. Time threshold 60 can be, for example, a predetermined time span after the closing of inspiration valve 20 or after the opening of expiration valve 22.

FIGS. 3a and 3b depict a flow signal profile 150, corresponding to what is depicted respectively in FIGS. 2a and 2b, for an ARDS patient. ARDS patients have a so-called “restructive” respiratory system whose compliance is very low. The respiratory system is less elastic than in respiratorily healthy patients. In COPD patients, conversely, the resistance of the respiratory system is elevated as compared with respiratorily healthy patients.

Because of the decreased compliance of the respiratory system of ARDS patients, the introduction of fresh respiratory gas into the patient's lungs requires greater work against the comparatively inelastic lungs and the likewise inelastic airways. Flow signal profile 150 is therefore fundamentally steeper than in respiratorily healthy patients, and most of all steeper than in COPD patients. The maximum flow magnitudes that are attained are also greater.

Details in FIGS. 3a and 3b that are identical and functionally identical to those in FIGS. 2a and 2b are labeled with the same reference characters, but incremented by 100.

As is evident from FIG. 3a, the expiratory respiratory gas flow reaches a higher magnitude at the beginning of the expiration phase than in the case of a COPD patient, a result of both the obstructive respiration of the COPD patient and the low compliance of the ARDS patient. For substantially the same reasons, the expiratory respiratory gas flow decreases more quickly in the ARDS patient, as shown in FIG. 3a, than in the COPD patient.

This is also made apparent by the fact that the expiratory time constant of an ARDS patient has a substantially lower value than that of the COPD patient.

Whereas for the COPD patient the flow signal profile proceeds at a constant slope over a long time period during the expiration phase, for the ARDS patient the slope of the flow signal profile usually changes several times during the expiration phase.

In order to evaluate the expiratory flow signal profile in terms of the presence of the trigger criterion that takes into account the change in the slope of the flow signal profile, for the trigger criterion under discussion here it is preferable to consider only those changes in the slope of flow signal profile 150 at which the slope of flow signal profile 150 increases, i.e. for which the curvature of flow signal profile 150 has a concave shape when viewed from the positive infinity of the ordinate, while concave changes in the slope, at which flow signal profile 150 becomes flatter, are not even considered.

Only those changes in the slope of flow signal curve 150 which are positive are therefore plotted in FIG. 3b.

It is apparent that a comparatively clear increase in slope during the expiration event at interpolation point 162 reaches a value that, for COPD patients, would be well beyond slope change threshold value 58 plotted in FIG. 2b and would thus initiate a transition from expiratory to inspiratory operation.

A switchover of ventilator 10 in this portion of the expiration phase would be very unpleasant for ARDS patients, since the patient would be confronted, in the middle of an expiration event, with an inspiration flow flowing against it.

Slope change threshold value 158 for ARDS patients is therefore advantageously selected to be greater than for COPD patients. A corresponding slope change threshold value for respiratorily healthy patients might lie between threshold values 58 and 158.

At the end of the expiration event, starting at interpolation point 154 the ARDS patient being supportively ventilated in PSV mode begins to aspirate respiratory gas, thus resulting in a considerable increase in the slope of flow signal profile 150. The profile portion between interpolation points 154 and 156 is considerably steeper, or exhibits a considerably greater slope, than the immediately preceding profile portion between interpolation points 152 and 154. Because of the independent inspiration effort of the ARDS patient who is spontaneously breathing at least occasionally during ventilation, this rise in the flow signal profile proceeds to the point of a positive flow signal. The associated change value 156s is shown in FIG. 3b. It exceeds slope change threshold value 158 defined for ARDS patients, and thus initiates a switchover from expiratory to inspiratory operation of ventilator 10.

A data memory of ventilator 10, in the form of a characteristics diagram, a table, a stored function or approximation function, and the like, can contain a correlation between the slope change threshold value to be applied in a ventilation instance and an expiratory time constant characterizing the state of the respiratory system of patient 12.

Control device 18 can then, proceeding from the expiratory time constant of patient 12 currently being ventilated, retrieve on the basis of the stored correlation the slope change threshold value to be applied to patient 12 currently being ventilated, or ascertain it via calculation operations.

The expiratory time constant (if known) of patient 12 currently being ventilated can be inputted manually into ventilator 10 by medical personnel, for example in order to have at least an initial value for the slope change threshold value.

Advantageously, control device 18 is embodied to ascertain, based on the sensor information accessible to it, the expiratory time constant of patient 12 who is respectively to be ventilated. Several measures for doing so are known in the existing art. One possible approach to ascertaining the expiratory time constant can be implemented based solely on the information obtained from flow sensor 44. For example, the expiratory time constant can be ascertained based on the respiratory gas volume exhaled during an expiration phase and the maximum respiratory gas flow that occurred during the same expiration phase. Further possible methods for ascertaining the expiratory time constant have already been noted above in the introduction to the description.

With ventilator 10 according to the present invention it is thus possible to ventilate, with high synchronicity in a low-impact and reliable manner, even patients having diametrically different pathological respiratory systems, since ventilator 10 is capable of ascertaining the state of the respiratory system of patient 12 and, proceeding from the ascertained state of the respiratory system, adapting to the respective patient who is to be ventilated the slope-dependent trigger criterion for switching ventilator 10 from expiratory to inspiratory operation.

1. A ventilator for at least supportive ventilation of living beings breathing in a healthy state, the ventilator comprising:
a connector configuration for connection to a respiratory gas reservoir;
a respiratory gas conduit arrangement conveying fresh inspiratory respiratory gas from the connector configuration to a patient interface upon inspiratory operation of the ventilator, and conveying metabolized expiratory respiratory gas away from the patient interface upon expiratory operation;
a pressure modifying arrangement for modifying the pressure of respiratory gas in the respiratory gas conduit arrangement;
a flow sensor that is embodied and arranged to furnish a flow signal representing a respiratory gas volumetric flow at least of expiratory respiratory gas in the respiratory gas conduit arrangement; and
a control device that controls the operation of the ventilator and is embodied to trigger a transition from expiratory operation to inspiratory operation of the ventilator when an increase in the slope of a flow signal profile, which reproduces flow signals furnished successively in time, exceeds a slope change threshold value,
wherein the control device (18) is further embodied to ascertain the slope change threshold value as a function of an expiratory time constant of the respective patient to be ventilated, and to trigger the transition from expiratory operation to inspiratory operation of the ventilator when the increase in the slope of the flow signal profile exceeds the slope change threshold value ascertained in accordance with the expiratory time constant.

2. The ventilator according to claim 1, wherein the control device is embodied to decrease the magnitude of the slope change threshold value with an increasing expiratory time constant, and vice versa.

3. The ventilator according to claim 1, wherein the control device is embodied to ascertain the expiratory time constant in one respective ventilation use instance.

4. The ventilator according to claim 3, wherein the control device is embodied to ascertain the expiratory time constant breath by breath.

5. The ventilator according to claim 3, wherein the control device is embodied to ascertain the expiratory time constant in accordance with the ratio between exhaled volume and the maximum expiratory volumetric flow that occurred in the context of that expiration event, and/or in accordance with the resistance and compliance that occur in the context of one breath.

6. The ventilator according to claim 1, wherein it comprises a data memory, at least readable by the control device, in which a correlation between the magnitude of the slope change threshold value and the expiratory time constant, or a correlation between a change value of the magnitude of the slope change threshold value and the expiratory time constant, is stored.

7. The ventilator according to claim 6, wherein a functional correlation between the magnitude of the slope change threshold value and the expiratory time constant, or a functional correlation between a change value of the magnitude of the slope change threshold value and the expiratory time constant, is stored in the data memory; and the control device is embodied to ascertain, proceeding from the expiratory time constant based on the functional correlation, a slope change threshold value that is to be used.

8. The ventilator according to claim 1, wherein the control device is embodied to smooth the flow signal furnished by the flow sensor, for example by low-pass filtering or by calculation of a moving average.

9. The ventilator according to claim 1, wherein the control device is embodied not to trigger the transition from expiratory operation to inspiratory operation of the ventilator until a predetermined time span has elapsed after a predefined characteristic event, for example a beginning of expiratory operation or a baseline-level crossing of the flow signal.

11. The ventilator according to claim 2, wherein the control device is embodied to ascertain the expiratory time constant in one respective ventilation use instance.

12. The ventilator according to claim 4, wherein the control device is embodied to ascertain the expiratory time constant in accordance with the ratio between exhaled volume and the maximum expiratory volumetric flow that occurred in the context of that expiration event, and/or in accordance with the resistance and compliance that occur in the context of one breath.

13. The ventilator according to claim 3, wherein the control device is embodied to ascertain the expiratory time constant, iteratively numerically, in accordance with the ratio between exhaled volume and the maximum expiratory volumetric flow that occurred in the context of that expiration event, and/or in accordance with the resistance and compliance that occur in the context of one breath.

14. The ventilator according to claim 4, wherein the control device is embodied to ascertain the expiratory time constant, iteratively numerically, in accordance with the ratio between exhaled volume and the maximum expiratory volumetric flow that occurred in the context of that expiration event, and/or in accordance with the resistance and compliance that occur in the context of one breath.
